(19) United States
(12) Patent Application Publication
(10) Pub. No.: US 2002/0120328 A1
(43) Pub. Date: Aug. 29, 2002

(54) Mechanical heart valve packaged in a liquid

(76) Inventors: Chandrashekhar Prabhakar Pathak, Austin, TX (US); Joseph Andrew Chinn, Austin, TX (US)

Correspondence Address: Timothy L. Scott, Senior Patent Counsel, SULZER MEDICA USA INC., Suite 1600, 3 East Greenway Plaza, Houston, TX, 77046, US

(21) Appl. No.: 09/746,380

(22) Filed: Dec. 21, 2000

(51) Int. Cl. A61F 2/24; A61B 19/02

(52) U.S. Cl. 623/2.2; 206/438

(57) ABSTRACT

A mechanical heart valve and packaging wherein the mechanical valve is surrounded by a liquid. In one embodiment, a glutaraldehyde solution is used. In another embodiment, organic solvents are used. The liquid will not cause a particulate to form during storage. In addition, the liquid will not produce harmful changes in the polymeric sewing ring, for example. That is, the liquid does not cause degradation of the polymeric materials comprising the heart valve, nor does it cause swelling of such materials, nor the dissolution of polymeric materials.

### TECHNICAL FIELD

The present invention pertains to prosthetic devices for implantation in a body of a patient and in particular to anti-bacterial packaging for mechanical prosthetic devices.

### BACKGROUND OF THE INVENTION

Prosthetic heart valves for human patients have been available since the 1950s, following the advent of blood oxygenators, which made open heart surgery possible. Today, there are two major types of heart valves: mechanical valves and bioprosthetic or tissue valves. The term “mechanical valve” as used herein, refers to a heart valve made exclusively of synthetic materials and which comprises essentially no biological components. The term “bioprosthetic valve,” on the other hand, refers to a heart valve comprising at least some biological components such as tissue or tissue components (e.g., collagen). The biological components are obtained from a donor animal (typically bovine or porcine), and the valve may comprise either biological materials alone or biological materials with man-made supports or stents. Early heart valve prostheses were exclusively mechanical valves. One such early design was the ball-and-cage valve, in which a ball or disc was housed in a cage. One side of the cage provided an orifice through which blood flowed either into or out of the heart, depending on which heart valve was being replaced. The energy of the blood flow in the forward direction forced the ball or disc to the back of the cage, allowing blood to flow through the valve. When blood attempted to flow in a reverse direction, or “regurgitate,” the energy of the blood flow forced the ball or disc into the orifice in the valve and blocked the flow of blood.

Bi-leaflet and monoleaflet mechanical heart valves were developed to overcome some of the deficiencies of early cage-based mechanical valve designs. A bi-leaflet valve typically comprises an annular valve body in which two opposed leaflet occluders are pivotally mounted. Monoleaflet heart valves typically comprise a single leaflet occluder coupled to the annular valve body. Monoleaflet valves typically open by pivoting movement, although some valves open by a combination of pivoting and translational movement. For both bi-leaflet and monoleaflet mechanical valves, the occluders are typically substantially rigid, although some designs incorporate flexible leaflets. In bi-leaflet valves, the leaflets move between a closed position in which the two leaflets are mated to prevent blood flow in the reverse direction, and an open position in which the occluders are pivoted away from each other to permit blood flow in the forward direction. In monoleaflet valves, the leaflet pivots and/or translates from the closed to the open position to allow blood flow. In each case, however, the energy of blood flow causes the occluders to move between their open and closed positions.

Mechanical heart valves are generally characterized by a rigid annular valve body supporting one or more occluders, with a sewing ring or sewing cuff circumscribing the annular valve body. Pyrolytic carbon is a material often used for the valve body or the occluders, although other materials such as metal, polymers or ceramics have also been proposed. The sewing ring is often comprised of silicone rubber with a polymeric fabric cover (e.g., Dacron TM fabric). A metal stiffening ring may be provided between the valve body and the sewing ring and a metal lock wire may be used to secure the stiffening ring and/or sewing ring to the valve body.

Mechanical valves have also been made with flexible leaflets fabricated from man-made materials such as polyurethane, silicone rubber or other biocompatible polymer, for example, a valve described by Purdy, et al., U.S. Pat. No. 5,562,729, incorporated herein by reference. A sewing ring is provided for mounting flexible leaflet mechanical heart valves in a patient's heart.

Bio-prosthetic heart valves, in contrast to mechanical valves, comprise an annulus formed by an annular stent to which three flexible leaflets, comprised of a biological material such as bovine or porcine pericardium, are coupled. When blood flows in the forward direction, the energy of the blood flow deflects the leaflets away from the center of the annulus and allows blood to flow in the forward direction. When the pressure across the valve reverses and blood begins to flow in the reverse direction, the three leaflets engage each other in a coaptive region, occluding the valve body annulus and preventing the flow of blood through the valve in the reverse direction. The valve leaflets are made from tissue, such as specially treated porcine or bovine pericardial tissue.

Mechanical heart valves have usually been packaged in containers that support the mechanical valve in such a way as to protect or isolate it from mechanical shocks. Representative packaging patents include Cromie, U.S. Pat. No. 4,101,031; Lubock et al., U.S. Pat. No. 4,801,015; Dohm et al., U.S. Pat. No. 5,720,391; and Caudillo et al., U.S. Pat. No. 5,823,342, all of which are hereby incorporated herein by reference in their entirety. Mechanical valves are typically shipped and stored in a sterilized condition in airtight containers. Because mechanical valves do not comprise biological materials, air is used as the medium in the containers. Inclusion of a liquid storage medium, such as an antibacterial solution, has been deemed unnecessary at best, and possibly damaging to the structural materials during storage, and has been avoided on the basis of added cost as well as the risk of possible harm to the valve. Bioprosthetic valves, on the other hand, are almost always shipped or stored in liquid media because of the need to maintain the biological components of the valve in a hydrated condition. In addition, the medium may have anti-bacterial properties or additives to ensure sterility and protect the biological components from bacterial degradation.

### BRIEF SUMMARY OF THE INVENTION

The present invention comprises a mechanical heart valve and packaging wherein the mechanical valve is surrounded by a liquid solution. The liquid acts as an additional physical barrier for preventing bacterial contamination of the valve, and is more efficient at deterring such contamination than current air-medium containers. Because prior art valves are not immersed in liquids, they are susceptible to bacterial contamination.

The liquid also has the ability to absorb mechanical vibration and shock better than containers without liquids. Accordingly, packaging in liquid further reduces the probability of mechanical damage in transporting and handling the valve. Pyrolytic carbon materials, commonly used in mechanical heart valves, are relatively brittle, and the additional dampening properties of a liquid medium can reduce the incidence of breakage during shipping.

The liquid in the container may have anti-microbial properties, which may include bacteriocidal and/or antifungal or antiviral agents. In preferred embodiments, the liquid is selected to be compatible with the materials used to fabricate the mechanical valve, so that the liquid will produce no harmful changes in the polymeric sewing ring, for example, or in flexible polymeric or silicone rubber leaflets, if used. That is, the solution preferably will not cause degradation, swelling, or dissolution of the polymeric or pyrolytic carbon materials comprising the heart valve. The liquid will not cause particles to form during storage. Such particles may be a polymer or a salt, for example. In one embodiment, the liquid comprises a gluteraldehyde solution. In another embodiment, organic solvents such as alcohol provide an improved capability for wetting hydrophobic surfaces such as Teflon TM material or polyester fabric.

These and other features and advantages of the invention will be apparent from the following description and accompanying drawings.

### BRIEF DESCRIPTION OF THE DRAWINGS

FIG. 1 is a perspective view of a container with a mechanical heart valve in a liquid.

FIG. 2 is an exploded perspective view of a container with a mechanical heart valve.

FIG. 3 is a cross sectional view of the container of FIG. 2 with mechanical heart valve in a sterile, anti-microbial liquid.

### DETAILED DESCRIPTION OF THE INVENTION

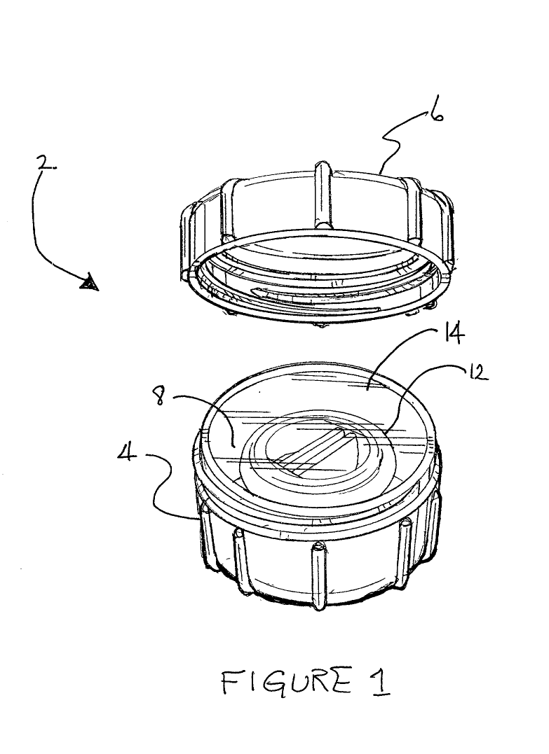

Containers incorporating a liquid solution for storage of a mechanical heart valve according to the present invention may be of any desired construction. While the Figures illustrate particularly preferred embodiments of the present invention, it is specifically contemplated that other embodiments of the invention, comprising either simpler or more complex container designs, are within the scope of the invention. FIG. 1 is a perspective view of an embodiment of the present invention illustrating a container 2 having a mechanical heart valve 12 therein, shown in phantom lines. The container 2 comprises a bottle 4 closed by a secure cap 6. Mechanical heart valve 12 is disposed within an interior space 8 in the container 2. Valve 12 is immersed in a liquid 14, which preferably has anti-microbial properties, as more fully described below. According to the teachings of the present invention, any suitable packaging may be used to contain the heart valve 12 and liquid 14. In the present embodiment, cap 6 is coupled to bottle 4 via a screwed connection. However, other coupling mechanisms, such as a snap-fit cap, can be used without departing from the scope of the invention. In addition, other types of containers may be used beyond bottles having caps. In particular, the invention may comprise a soft polymeric pouch package having an interior space comprising a valve (such as valve 12) and a liquid (such as liquid 14) therein.

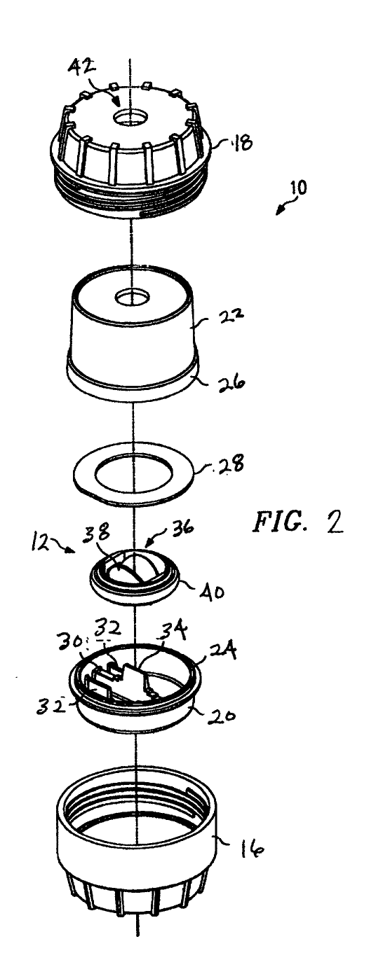

FIG. 2 is an exploded view of another embodiment of packaging 10 for a mechanical heart valve 12, to which a liquid could be added in accordance with the present invention. In the illustrated embodiment, packaging 10 includes an outer shell comprising a first outer shell portion 16 and a second outer shell portion 18. First outer shell portion 16 and second outer shell portion 18 are formed to removably engage one another. In the illustrated embodiment, the outer shell portions 16, 18 can be formed with threads such that the two portions can be screwed together. Other coupling means could be used without departing from the scope of the invention.

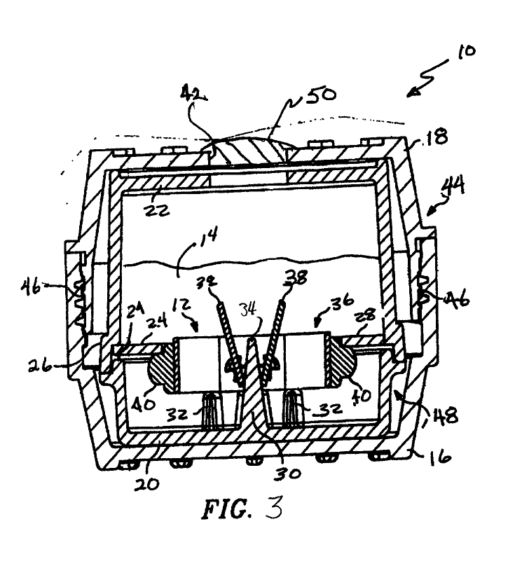

As shown in FIGS. 2 and 3, the outer shell forms a housing for a container 48 that has a first container portion 20 and a second container portion 22. The container portions 20, 22 are formed to removably engage one another. In particular, container portion 20 has a perimeter ridge 24 sized to fit inside a perimeter rim 26 of container portion 22. Container portion 20 and container portion 22, when engaged, have inner surfaces that define an inner compartment for housing the mechanical heart valve 12. Container portion 20 provides support members for a heart valve comprising a shelf 28, central ridge 30, tab 34, and lateral ridges 32. These members together engage and support heart valve 12.

Shelf 28 is sized to fit inside and removably engage (e.g., press fit) rim 26 of the container portion 22. A support member such as shelf 28 may also be formed integrally with container portion 22. Container portion 20 further provides opposed support members that cooperate with shelf 28, specifically a central ridge 30 and a pair of lateral ridges 32. The central ridge 30 and lateral ridges 32 can be coupled to, or formed integrally with, the inner surface of container portion 20. Further, central ridge 30 can include a tab 34 that extends into the inner compartment formed when container portions 20 and 22 are engaged. As shown in FIG. 3, central ridge 30, lateral ridges 32, and tab 34 engage heart valve 12 opposite shelf 28 to provide support to valve 12 in container 48. In the embodiment of FIGS. 2 and 3, central ridge 30 and lateral ridges 32 are formed integral with container portion 20. However, other implementations are also possible, for example, where the support member is a separate component from container portion 20.

Mechanical heart valve 12 is representative of the set of implantable medical devices suitable for use with the present invention. Such devices include mechanical heart valves with flexible polymeric or silicone rubber leaflets, such as the heart valve of Purdy et al., U.S. Pat. No. 5,562,729; vascular grafts, such as the grafts of Lauterjung U.S. Pat. No. 5,824,036 or Lauterjung WO97/48350 (both incorporated herein by reference in their entirety) or angioplasty rings, such as the Campbell ring, U.S. Pat. No. 6,102,945 (incorporated herein by reference in their entirety), constructed from non-biological materials.

The mechanical heart valve 12, illustrated as a bi-leaflet valve in the embodiment of FIG. 2, typically comprises an orifice 36 to which leaflets 38 are pivotally coupled. Alternatively, flexible polymeric or silicone rubber leaflets may act as occluders, as described in U.S. Pat. No. 5,562,729. Mechanical heart valve 12 preferably comprises a sewing cuff 40 that is used to affix the mechanical heart valve 12 to the patient's heart. The mechanical heart valve 12 can operate as a mitral or aortic heart valve when implanted in a human heart, depending upon its orientation when implanted. To insure that mechanical heart valve 12 can be sterilized, a hole 42 is provided in packaging 10 to allow sterilization of the inside of package 10. Although numerous modifications and design choices may be employed by persons of skill in the art, packaging 10 is easy to manufacture and assemble by using components that are standard across the mechanical heart valve product line.

When the heart valve is to be implanted, the outer shell of packaging 10 is opened, and the inner container can then be removed and positioned in the aortic or mitral orientation. In the aortic orientation, container portion 20 is positioned at the bottom with container portion 22 on top. Container portion 22 can then be removed along with shelf 28. This leaves mechanical heart valve 12 supported by the aortic support member formed by central ridge 30 and lateral ridges 32 with leaflets 38 held open by tab 34. A holding instrument (not shown) can then be used to extract mechanical heart valve 12 from packaging 10 and hold it for implantation in a surgical procedure. Tab 34 holds leaflets 38 open to ensure that the holding instrument can be used without having to manually manipulate leaflets 38. Thus, in the aortic orientation, the mechanical heart valve 12 is supported such that it is prepared for receiving a holding instrument for implantation as an aortic valve.

FIG. 3 is a cross-section view of the packaging 10 of FIG. 2 after assembly. As shown, an outer shell, indicated generally at 44, is formed by the engaging of outer shell portion 18 and outer shell portion 16 using threads 46. Container 48 is housed inside outer shell 44 and is formed by a first container portion 20 and a second container portion 22. In this embodiment, as shown, ridge 24 of container portion 20 fits inside rim 26 of container portion 22. In the aortic orientation of FIG. 2, the mechanical heart valve 12 rests on central ridge 30 and lateral ridges 32 with leaflets 38 held open by tab 34 on ridge 30. As can be seen from FIG. 3, space in orifice 36 receives a holding device due to tab 30 holding leaflets 38 in an open position.

After packaging 10 has been assembled around the valve 12, the valve and packaging can optionally be steam sterilized through the hole 42. The method of sterilization is not critical, however, and other sterilization techniques known in the art may be used. Before or after sterilization, depending upon the sterilization technique employed, liquid 14 is preferably poured through the hole 42, to a level sufficient to cover the valve. As persons of skill in the art will readily appreciate, any desired method of introducing the liquid 14 into the valve may be employed. After the liquid is introduced into the packaging 10, the hole 42 is then sealed with a plug 50 to provide a sealed, leakproof container for heart valve 12.

In addition to optionally having anti-microbial characteristics, the selected liquid 14 should preferably avoid forming precipitates or particles throughout a wide range of extreme conditions that may be encountered, particularly during shipping and storage. In preferred embodiments, the liquid should be able to withstand temperatures between −8° C. and 40° C. in the presence of materials comprising the mechanical heart valve and container without formation of a precipitate. Moreover, the liquid 14 should not adversely affect any of the materials comprising the mechanical heart valve, that is, the fluid should not affect the size, weight, dimensions or visual appearance of the materials. Mechanical heart valves are generally comprised of three types of materials: polymeric components, such as silicone rubber or polyurethane in the sewing ring; metal components, such as stiffening rings or lock rings; and pyrolytic carbon components, such as leaflets or an annular valve body. Various anti-bacterial liquids were tested for these criteria, as described in connection with Tables 1 and 2, below.

Gluteraldehyde solution is a suitable anti-bacterial storage medium. An appropriate concentration is believed to be between 0.1% and 50%, more preferably between 0.2% and 0.6%. In certain storage media, it was determined that the pH should be controlled to inhibit the formation of particles, or deposition of a precipitate. An inorganic buffer such as phosphate buffer may be used. Because of the materials used in manufacturing mechanical heart valves, however, an organic buffer, such as HEPES buffer or triethanol amine buffer, may be used. Preferably, the pH should be kept between pH 7.0 and pH 7.4.

Selected liquids were stored for twenty hours at −4° C. The solutions were then observed for precipitate or particle formation. Precipitation was present in 50% ethanol, 50% 20 mM phosphate buffered saline and in gluteraldehyde solution at pH 10.0. On the other hand, solutions of 70% isopropanol; 50% ethanol; 0.2% gluteraldehyde in HEPES or PBS (that is, at physiological pH); and acetone (10% and 100%) showed no precipitate, and were candidates for further testing. The results of this series of tests are summarized in the following Table 1.

TABLE 1
Test for Precipitation at −4° C.

| Test Solution | Observation after 20 hours at −4° C. |
|---|---|
| 70% Isopropanol and 30% distilled | No change |
| H₂O | |
| 50% Ethanol and 50% 20 mM | Large crystalline precipitate |
| phosphate buffered saline | |
| 50% ethanol and 50% 10 mM | No change |
| triethanol amine buffer | |
| 0.2% gluteraldehyde solution with | No change |
| HEPES buffer at pH 7.2 | |
| 0.2% gluteraldehyde solution with | No change |
| 20 mM phosphate buffered saline | |
| 10% gluteraldehyde solution in | Thick milky precipitate |
| distilled H₂O, pH adjusted | |
| to pH 10 using 3 M NaOH | |
| 10% acetone | No change |
| 100% acetone | No change |

Those solutions that passed Test 1 above were tested for effects on heart valve materials. Individual components of mechanical valves from the three categories described above were stored in solutions for 11 days. Dry weight change is believed to be representative of actual change in components. The effect of storage in selected solutions is summarized in the following Table 2.

TABLE 2
Dry Weight Change of Mechanical Valve Components after Storage

| | | Pre-weight | Post-weight | |
|---|---|---|---|---|
| Solution | Component | (g) | (g) | % Change |
| 70% IPA | Metal ring | 2.1099 | 2.1099 | 0.000 |
| | Pyrolite | 0.4509 | 0.4508 | −0.022 |
| | Silicone | 0.1343 | 0.1339 | −0.298 |
| | Suture | 0.0334 | 0.0336 | 0.599 |
| | Teflon | 0.079 | 0.079 | 0.000 |
| | Fabric | 0.0343 | 0.0339 | −1.166 |
| 0.2% Glut/PBS | Metal ring | 2.1043 | 2.1044 | 0.005 |
| | Pyrolite | 0.4584 | 0.4586 | 0.044 |
| | Silicon | 0.1481 | 0.1482 | 0.068 |
| | Suture | 0.0332 | 0.0334 | 0.602 |
| | Teflon | 0.0956 | 0.0954 | −0.209 |
| | Fabric | 0.0362 | 0.0362 | 0.000 |
| 0.2% Glut/HEPES | Silicon | 0.1434 | 0.1438 | 0.279 |
| | Suture | 0.0082 | 0.0081 | −1.220 |
| | Teflon | 0.0883 | 0.0878 | −0.566 |
| | Fabric | 0.0425 | 0.0426 | 0.235 |
| 50% EtOH/TEA | Metal ring | 0.0591 | 0.0594 | 0.508 |
| | Silicon | 0.1428 | 0.1426 | −0.140 |
| | Suture | 0.0075 | 0.0076 | 1.333 |
| | Teflon | 0.0971 | 0.0972 | 0.103 |
| | Fabric | 0.039 | 0.0389 | −0.256 |
| 100% Acetone | Silicon | 0.1334 | 0.1303 | −2.324 |
| | Suture | 0.0073 | 0.0076 | 4.110 |
| | Teflon | 0.0903 | 0.09 | −0.332 |
| | Fabric | 0.0436 | 0.0401 | −8.028 |

For the selected solutions, all the components remained within 2% of their original weight after storage with the exception of 100% acetone. In connection with this test, the size, visual appearance and dimensions of each component were measured or examined before and after storage. No significant changes were noted. The solutions identified in Table 2, with the exception of 100% acetone, are considered to be appropriate sterile liquid storage media for mechanical heart valves.

The foregoing describes preferred embodiments of the invention and is given by way of example only. The invention is not limited to any of the specific features described herein, but includes all variations thereof within the scope of the appended claims.

What is claimed is:

1. A combination of a mechanical heart valve and packaging comprising
a mechanical heart valve having
an annular valve body having an inside surface and an outside surface,
a leaflet occluder coupled to said valve body, and
a sewing ring attached to said outside surface of said annular valve body,
a sealed container, said mechanical heart valve being disposed within in said container, and
a liquid disposed within said container, said mechanical heart valve being immersed in said solution.

2. The combination of claim 1 wherein said liquid comprises a solution consisting of between 0.1% and 50% gluteraldehyde.

3. The combination of claim 2 wherein said liquid consists of between 0.2 and 0.6% gluteraldehyde.

4. The combination of claim 1 wherein said liquid has a pH sufficient to prevent formation of particles.

5. The combination of claim 4 wherein said liquid is buffered.

6. The combination of claim 5 wherein said liquid comprises an organic buffer.

7. The combination of claim 6 wherein said organic buffer is selected from the group consisting of: HEPES and triethanol amine buffer.

8. The combination of claim 5 wherein said liquid is buffered with an inorganic buffer.

9. The combination of claim 8 wherein said liquid is buffered with phosphate buffer.

10. The combination of claim 4 wherein said liquid has a pH less than 7.4.

11. The combination of claim 1 wherein said liquid comprises from 20% to 100% alcohol.

12. The combination of claim 1 wherein said heart valve further comprises a polymer and wherein said liquid inhibits at least one of degradation of said polymeric material, swelling of said polymeric material, and dissolution of said polymeric material.

13. A combination of an implantable medical device and packaging comprising an implantable medical device having no tissue components and further comprising
a polymeric fabric element configured to contact tissue of a patient in whom the device is to be implanted, and
a sealed container, said device being placed in said container, and
an anti-microbial solution in said container, said device being in contact with said solution.

14. The combination of claim 13 wherein said device is selected from a group consisting of a mechanical heart valve, a vascular graft, an annuloplasty ring, or a combination thereof.

15. The combination of claim 13 wherein said solution comprises a solution consisting of greater than 0.2% gluteraldehyde.

16. The combination of claim 13 wherein said solution has a pH sufficient to prevent formation of a particulate.

17. The combination of claim 16 wherein said solution has a pH less than 7.4.

18. The combination of claim 16 wherein said solution comprises at least 20% alcohol.

19. The combination of claim 18 wherein said solution inhibits change of said polymeric fabric element.
